SEQUENCE LISTING (5 sequences)

SEQ ID NO: 1 - protein, 42 aa, Homo sapiens [sequence omitted]

SEQ ID NO: 2 - protein, 404 aa, Homo sapiens
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 3 - protein, 32 aa, Artificial Sequence
  Synthetic Construct
MGTNLSVPNPLGFLPDHQLDPGGVQGEESNDK

SEQ ID NO: 4 - protein, 24 aa, Homo sapiens
CTTPAQGNSMFPSCCCTKPTDGNC

SEQ ID NO: 5 - protein, 32 aa, Homo sapiens
AQNITARIGEPLVLKCKGAPKKPPQRLEWKLN

(19) United States
(12) Patent Application Publication
(10) Pub. No.: US 2010/0297160 A1
(43) Pub. Date: Nov. 25, 2010

(54) High Molecular Weight Amyloid Beta As a Carrier for the Oral Delivery of Vaccine Antigens

(75) Inventors: Shyamala Mruthinti, Evans, GA (US); Michael Bartlett, Bogart, GA (US)

Correspondence Address: Pabst Patent Group LLP, 1545 PEACHTREE STREET NE, SUITE 320, ATLANTA, GA, 30309, US

(73) Assignee: Medical College of Georgia Research Institute, Inc.

(21) Appl. No.: 12/780,628

(22) Filed: May 14, 2010

Related U.S. Application Data
(60) Provisional application No. 61/216,192, filed on May 14, 2009.

(51) Int. Cl. A61K 39/00; A61P 25/28; C12N 5/02

(52) U.S. Cl. 424/185.1; 424/184.1; 435/375

(57) ABSTRACT

Compositions and methods are provided for stabilizing polypeptide antigens such as amyloid-beta (Aβ) to produce vaccines for oral delivery. One embodiment provides an immunogenic polypeptide complex of Aβ₄₂ and an fragment of receptor for advanced glycation endproducts (RAGE).

### CROSS REFERENCE TO RELATED APPLICATIONS

This application claims benefit of U.S. Provisional Application No. 61/216,192, filed May 14, 2009, which is hereby incorporated herein by reference in its entirety.

### FIELD OF THE INVENTION

The invention is generally related to the field of vaccines, more particularly to methods and compositions for preparing an oral vaccine for Alzheimer's disease.

### BACKGROUND OF THE INVENTION

Alzheimer's disease (AD) is the most common form of dementia. AD affects as many as 4.5 million Americans impacting many normal daily activities by degrading parts of the brain that control thought, memory, and language. It is anticipated that the prevalence of AD will grow over the next four decades becoming the leading cause of death in North America by 2050 (Trojanowski, J Q. Neurosignals 16: 5-10, 2008). Although more is known every day concerning AD currently there is no cure.

AD is named for Dr. Alois Alzheimer. In 1906, Dr. Alzheimer noticed changes in the brain tissue of a woman who had died of an unusual mental illness. He found abnormal clumps (now called amyloid plaques) and tangled bundles of fibers (now called neurofibrillary tangles). Today, these plaques and tangles in the brain are considered the definitive sign of AD.

There are three major competing hypotheses that exist to explain the cause of the disease. The oldest, on which most currently available drug therapies are based, is known as the cholinergic hypothesis and suggests that AD is due to reduced biosynthesis of the neurotransmitter acetylcholine. However, the medications that treat acetylcholine deficiency only affect symptoms of the disease and neither halt nor reverse it (Walker and Rosen, Age and Aging 35(4): 332-35, 2006). The cholinergic hypothesis has not maintained widespread support in the face of this evidence, although cholinergic effects have been proposed to initiate large-scale aggregation and generalized neuroinflammation (Perry et al., British Medical Journal 2(6150): 1457-1459, 1978).

The tau hypothesis states that abnormalities in the tau protein initiate the disease cascade (Takashima et al., PNAS 90: 7789-93, 1993; Rapoport et al., PNAS 90: 7789-93, 2002). The tau hypothesis is supported by the long-standing observation that deposition of amyloid plaques does not correlate well with neuron loss.

In 1991, the amyloid hypothesis was proposed which states that amyloid beta (Aβ) deposits are the causative agent in the disease (Hardy J, Allsop D, Trends in Pharmacol. Sci. 12(10): 383-88, 1991; Hardy J A, Higgins G A, Science 256: 184-85, 1992). The amyloid hypothesis is compelling because the gene for the amyloid beta precursor protein (APP) is located on chromosome 21, and patients with Down Syndrome who thus have an extra gene copy almost universally exhibit AD-like disorders by age 40 (Lott et al., Neurobiol. of Aging 26(3): 383-89, 2005; Nistor et al., Neurobiol. of Aging 28(10): 1493-1506, 2007). The traditional formulation of the amyloid hypothesis points to the cytotoxicity of mature aggregated amyloid fibrils, which are believed to be the toxic form of the protein responsible for disrupting cellular calcium ion homeostasis and thus inducing apoptosis. It should be noted further that ApoE4, the major genetic risk factor for AD, leads to excess amyloid build-up in the brain before AD symptoms arise (Polvikoski et al., New England Journal of Medicine 333(19): 1242-47, 1995). Thus, Aβ deposition precedes clinical AD. Another strong support for the amyloid hypothesis, which looks at Aβ as the common initiating factor for Alzheimer's disease, is that transgenic mice solely expressing a mutant human APP gene (the PDAAP mouse model) has been shown to develop fibrillar amyloid plaques (Games et al., Nature 373: 523-27, 1995).

Importantly, immunization with amyloid-beta (Aβ) attenuates Alzheimers-disease pathology in a transgenic Alzheimer's mouse model (Schenk, D, et al. Nature 400(6740):173-7, 1999). However, Aβ alone was not sufficiently immunogenic since it is an endogenous protein; therefore in order to induce an immune response the co-administration of an adjuvant was required. This discovery rapidly led to the initiation of the AN-1792 vaccine trial in 2000. This vaccine consisted of the Aβ peptide and an adjuvant QS-21 that stimulated the uptake of the Aβ peptide by antigen-presenting cells. The early Phase I trials with AN-1792 continued to demonstrate promise for this approach. However, the subsequent Phase II trial was suspended when patients reported serious inflammation in the brain. Despite the trial ending prematurely, approximately 20% of these patients developed high levels of antibodies against the Aβ peptide and demonstrated improved performance in memory-tests.

Currently there are two major Alzheimer's vaccine trials underway. The first is the AAC-001 trial which is a modified approach to the earlier AN-1792 vaccine. AAC-001 uses an adeno-associated viral vector to deliver Aβ DNA to the antigen producing cells to elicit the immune response. The AAC-001 trial does not use an adjuvant as this was believed to be the cause of the inflammation that ended the AN-1792 trial. However, the AAC-001 trial was also suspended in early April 2008 when a single patient developed a severe skin inflammation.

AAB-001 uses a different approach than the previous trials which delivered the Aβ antigen to produce an immune response. The AAB-001 trial directly delivers a monoclonal antibody (bapineuzumab) against the Aβ peptide. This approach is known as passive immunotherapy because it does not actively invoke an immune response in the patient and therefore requires regular infusion to maintain its effectiveness.

It is therefore an object of the invention to provide a safe, stable, and effective Alzheimer's vaccine. For example, it is an object of the invention to provide an oral vaccine that is stable at ambient temperatures.

### SUMMARY OF THE INVENTION

Compositions and methods are provided for stabilizing polypeptide antigens such as amyloid-beta (Aβ) to produce vaccines for oral delivery. One embodiment provides an immunogenic polypeptide complex of Aβ₄₂ and an fragment of receptor for advanced glycation endproducts (RAGE). Another embodiment provides a method of provoking an immune response in a lymphocyte, involving contacting the lymphocyte with the disclosed immunogenic polypeptide complex. Another embodiment provides a pharmaceutical composition involving an effective amount of the disclosed immunogenic polypeptide complex in a pharmaceutically acceptable excipient. Another embodiment provides a method of treating Alzheimer's disease in a subject, involving administering to the subject the disclosed pharmaceutical composition.

### BRIEF DESCRIPTION OF THE DRAWINGS

FIG. 1 is a graph showing IgG titers for anti-RAGE (circle), anti-Aβ (square), and anti-RAGE-Aβ complex (triangle) as a function of fold dilution of plasma from rats immunized with orally-administered RAGE-Aβ complex. Each data point represents mean±S.E.M., n=3.

FIG. 2 is a three-dimensional (3D) graph showing concentration of plasma Aβ in double AD Tg mice (B6C3-Tg(APPswe,PSEN1dE9)85Dbo/J) immunized with Aβ-RAGE complex as a function of age (3, 6, 12, and 18 months) and length of experiment (weeks).

FIG. 3 is a three-dimensional (3D) graph showing concentration of plasma RAGE in AD Tg mice immunized with Aβ-RAGE complex as a function of age (3, 6, 12, and 18 months) and length of experiment (weeks).

FIG. 4 is a bar graph showing brain anti-Aβ (right set of bars) and anti-RAGE (left set of bars) IgG concentration as a function of age (3, 6, 12, and 18 months) in immunized (right bars) and non-immunized (left bars) AD Tg mice.

FIG. 5 is a bar graph showing whole brain soluble Aβ (right set of bars) and soluble RAGE (right set of bars) protein concentration as a function of age (3, 6, 12, and 18 months) in immunized (right bars) and non-immunized (left bars) AD Tg mice.

FIG. 6a is a graph showing the concentration of plasma anti-RAGE IgG (grey circles, left axis) and plasma anti-Aβ IgG (black circles, right axis) in macaque monkeys as a function of monkey age. FIG. 6b is a bar graph showing plasma anti-RAGE IgG (grey bars) and plasma anti-Aβ IgG (black bars) derived from 8-11 wild type (WT, first and third bars) and AD transgenic (Tg (APPSWE/PS1), second and fourth bars) mice 8-12 months old. FIG. 6c is a set of bar graphs showing plasma anti-RAGE IgG (right graph) and plasma anti-Aβ IgG (left graph) derived from study participants as a function of diagnosis, i.e., control elderly participants (left bars), participants with mild cognitive impairment (MCI, middle bars), and participants with Alzheimer's disease (right bars).

FIG. 7 is a set of graphs showing anti-RAGE IgG (top graph) and plasma anti-Aβ IgG (bottom graph) as a function of antigen exposure (_{H}/m1/5 days) in human peripheral blood mononuclear cells (PBMCs) culture for Aβ₄₂ (black circles), sRAGE (grey circles), and Aβ-RAGE complex (black triangles).

### DETAILED DESCRIPTION OF THE INVENTION

The present disclosure provides a delivery strategy for oral vaccines using a polypeptide complex. This strategy can be applied using peptides such as Aβ₄₂ but can be expanded to other peptides that can form a complex, induce antibody production and possess appropriate stability in the gastrointestinal tract.

### I. Definitions

The term “polypeptide” refers to a chain of amino acids of any length, regardless of modification (e.g., phosphorylation or glycosylation). The polypeptide is not limited by length; thus “polypeptide” can include peptide, oligopeptide, gene product, expression product, or protein.

The term “isolated polypeptide” refers to a polypeptide (or a fragment thereof) that is substantially free from the materials with which the polypeptide is normally associated in nature. The disclosed polypeptides, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides, including natural or synthetic polypeptides.

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid. Conservative substitutions and deletions are described below.

The term “residue” or “position,” with respect to an amino acid residue in a polypeptide, refers to a number corresponding to the numerical place that residue holds in the polypeptide. By convention, residues are counted from the amino terminus to the carboxyl terminus of the polypeptide. Thus, position 42 of human Aβ would be the 42nd residue from the amino terminus of the Aβ protein sequence.

The term a “variant” polypeptide refers to a polypeptide that contains at least one amino acid sequence alteration as compared to the amino acid sequence of the corresponding wild-type polypeptide.

The term “amino acid sequence alteration” refers to, for example, a substitution, a deletion, or an insertion of one or more amino acids.

The term “conservative variant” refers to one or more conservative amino acid substitutions or deletions.

The term “nucleic acid” refers to a natural or synthetic molecule having a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “isolated nucleic acid” refers to a nucleic acid that is separated from other nucleic acid molecules that are present in a mammalian genome, including nucleic acids that normally flank one or both sides of the nucleic acid in a mammalian genome.

The term “vector” refers to a replicon, such as a plasmid, phage, or cosmid, into which another DNA segment may be inserted so as to bring about the replication of the inserted segment. The vectors described herein can be expression vectors.

The term “expression vector” refers to a vector that includes one or more expression control sequences

The term an “expression control sequence” refers to a DNA sequence that controls and regulates the transcription and/or translation of another DNA sequence.

The term “operably linked” means incorporated into a genetic construct so that expression control sequences effectively control expression of a coding sequence of interest.

The term “fragment” of a polypeptide refers to any subset of the polypeptide that is a shorter polypeptide of the full length protein. Generally, fragments will be five or more amino acids in length.

The term “valency” refers to the number of binding sites available per molecule.

The term “conservative” amino acid substitutions refer to substitutions wherein the substituted amino acid has similar structural or chemical properties.

The term “non-conservative” amino acid substitutions refers to those in which the charge, hydrophobicity, or bulk of the substituted amino acid is significantly altered.

The term “cell” refers to individual cells, cell lines, primary culture, or cultures derived from such cells unless specifically indicated.

The term “culture” refers to a composition having isolated cells of the same or a different type.

The term “cell line” refers to a culture of a particular type of cell that can be reproduced indefinitely, thus making the cell line “immortal.”

The term “host cell” refers to prokaryotic and eukaryotic cells into which a recombinant expression vector can be introduced.

The term “transformed” and “transfected” encompass the introduction of a nucleic acid (e.g., a vector) into a cell by a number of techniques known in the art.

The term “antibody” is meant to include both intact molecules as well as fragments thereof that include the antigen-binding site. These include Fab and F(ab′)₂ fragments which lack the Fc fragment of an intact antibody.

The terms “individual”, “host”, “subject”, and “patient” are used interchangeably herein, and refer to a mammal, including, but not limited to, humans, rodents, such as mice and rats, and other laboratory animals.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

The term “pharmaceutically acceptable” refers to a material that is not biologically w otherwise undesirable, i.e., the material may be administered to a subject, along with the disclosed polypeptide, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The term “peptidomimetic” means a mimetic of a peptide which includes some alteration of the normal peptide chemistry.

The term “liposome” refers to a structure having an outer lipid bi- or multi-layer membrane surrounding an internal aqueous space.

The term “immunogenic” or “immunogenicity” refers to the ability of a particular substance, such as an antigen or epitope, to provoke humoral and/or cell-mediated immune response in a subject. This includes the ability of a disclosed polypeptide to simulate in the subject the secretion of antibodies that specifically bind the polypeptide by B lymphocytes.

### II. Compositions

Full length soluble RAGE (sRAGE) complexed with Aβ₄₂ is a natural complex found in human plasma. RAGE-Aβ interaction is the RAGE-dependent transport of Aβ from blood to brain. A consequence of the RAGE-Aβ interaction is the formation of a soluble high molecular weight complex of the two proteins that is both neurotoxic and immunogenic. It is demonstrated here that this immunogenicity leads to the formation of circulating endogenous antibodies titers with dual affinity for both the RAGE and Aβ peptides. Aβ and sRAGE are not particularly immunogenic peptides. However, they can complex with each other producing a new species that is immunogenic and for which endogenous antibodies are induced.

Thus, disclosed herein are immunogenic polypeptides that can be combined to form stable polypeptide complex (e.g., aggregate) for use as oral vaccines. The immunogenic polypeptide complex can be formed using covalent or ionic bonds.

In a preferred embodiment, the immunogenic polypeptide complex has at least a first and second polypeptide. The first polypeptide is preferably Aβ₄₂. The second polypeptide is preferably an immunogenic fragment of RAGE. For example, the second polypeptide can be amino acids 23 to 54 of human RAGE. The disclosed examples demonstrate that an Aβ₄₂-RAGE₂₃₋₅₄ complex is stable and effective as an oral vaccine. Full length soluble RAGE (sRAGE) also can be co-incubated with Aβ₄₂ to form immunogenic high molecular weight (>150 kDa) complex protein.

A. Amyloid Beta

Amyloid beta (Aβ) is formed after sequential cleavage of the amyloid precursor protein (APP), a transmembrane glycoprotein of undetermined function. Aβ protein is generated by successive action of β- and γ-secretases on APP. The γ-secretase, which produces the C-terminal end of the Aβ peptide, cleaves within the transmembrane region of APP and can generate a number of isoforms of 39-43 amino acid residues in length. The most common isoforms are Aβ₄₀ and Aβ₄₂; the shorter form is typically produced by cleavage that occurs in the endoplasmic reticulum, while the longer form is produced by cleavage in the trans-Golgi network. The Aβ₄₀ form is the more common of the two, but Aβ₄₂ is the more fibrillogenic and is thus associated with disease states. Mutations in APP associated with early-onset Alzheimer's have been noted to increase the relative production of Aβ₄₂, and thus one suggested avenue of Alzheimer's therapy involves modulating the activity of β- and γ-secretases to produce mainly Aβ₄₀.

Thus, the disclosed immunogenic polypeptide complex can include Aβ₄₂. An amino acid sequence for human Aβ₄₂ is set forth in SEQ ID NO:1, shown below:

DAEFRHDSGY EVHHQKLVFF AEDVGSNKGA IIGLMVGGVV IA.

Thus, the disclosed immunogenic polypeptide complex can include a first polypeptide having the amino acid sequence SEQ ID NO:1, or a conservative variant thereof.

It is also understood that the skilled artisan can identify similar Aβ proteins from other species using routine skill with a high expectation that these sequences will retain the ability to bind the immunogenic fragment of RAGE and be immunogenic in human.

Another embodiment of the immunogenic polypeptide complex provides a first polypeptide having an amino acid sequence that is at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:1, wherein the polypeptide binds RAGE₂₃₋₅₄ under physiological conditions. Thus, the first polypeptide can have an amino acid sequence that is at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:1, wherein the polypeptide binds RAGE₂₃₋₅₄ under physiological conditions.

B. RAGE

The disclosed immunogenic polypeptide complex can include an immunogenic fragment of the receptor for advanced glycation endproducts (RAGE). RAGE is a 35 kD transmembrane receptor of the immunoglobulin super family. Its name comes from its ability to bind advanced glycation endproducts (AGE), a heterogeneous group of non-enzymatically altered proteins. Besides AGEs, RAGE is also able to bind other ligands and is thus often referred to as a pattern recognition receptor.

The interaction between RAGE and its ligands is thought to result in pro-inflammatory gene activation. Due to an enhanced level of RAGE ligands in diabetes or other chronic disorders, this receptor is hypothesized to have a causative effect in a range of inflammatory diseases such as diabetic complications, Alzheimer's disease and even some tumors.

Isoforms of the RAGE protein, which lack the transmembrane and the signaling domain (commonly referred to as soluble RAGE or sRAGE) are hypothesized to counteract the detrimental action of the full-length receptor and are hoped to provide a means to develop a cure against RAGE-associated diseases.

The primary transcript of the human RAGE gene is thought to be alternatively spliced. So far about 6 isoforms including the full length transmembrane receptor have been found in different tissues such as lung, kidney, brain etc. Five of these 6 isoforms lack the transmembrane domain and are thus believed to be secreted from cells. Generally these isoforms are referred to as sRAGE (soluble RAGE) or esRAGE (endogenous secretory RAGE). One of the isoforms lacks the V-domain and is thus believed not to be able to bind RAGE ligands.

The full receptor consists of 5 domains: The cytosolic domain, which is responsible for signal transduction, the transmembrane domain which anchors the receptor in the cell membrane, the variable domain which binds the RAGE ligands and two constant domains.

RAGE is able to bind several ligands and therefore is referred to as a pattern-recognition receptor. Proteins which have so far been found to bind RAGE are AGE, HMGB1 (Amphoterin), S100b. Aβ protein, and Mac-1.

Thus, disclosed for use in the disclosed compositions and methods is an immunogenic fragment of RAGE. For example, the immunogenic fragment of RAGE can be amino acids 23 to 54 of human RAGE. The amino acid sequence for human RAGE is set forth in SEQ ID NO:2, shown below:

MAAGTAVGAW VLVLSLWGAV VGAQNITARI GEPLVLKCKG
APKKPPQRLE WKLNTGRTEA WKVLSPQGGG PWDSVARVLP
NGSLFLPAVG IQDEGIFRCQ AMNRNGKETK SNYRVRVYQI
PGKPEIVDSA SELTAGVPNK VGTCVSEGSY PAGTLSWHLD
GKPLVPNEKG VSVKEQTRRH PETGLFTLQS ELMVTPARGG
DPRPTFSCSF SPGLPRHRAL RTAPIQPRVW EPVPLEEVQL
VVEPEGGAVA PGGTVTLTCE VPAQPSPQIH WMKDGVPLPL
PPSPVLILPE IGPQDQGTYS CVATHSSHGP QESRAVSISI
IEPGEEGPTA GSVGGSGLGT LALALGILGG LGTAALLIGV
ILWQRRQRRG EERKAPENQE EEEERAELNQ SEEPEAGESS
TGGP .

Thus, the immunogenic fragment of RAGE can have the amino acid sequence SEQ ID NO:5, shown below:

AQNITARIGE PLVLKCKGAP KKPPQRLEWK LN.

The immunogenic fragment of RAGE can have an amino acid sequence having at least 95% sequence identity to SEQ ID NO:5. Thus, the immunogenic fragment of RAGE can have a conservative variant of the amino acid sequence SEQ ID NO:5.

C. Combinations

Also disclosed are compositions having one or more disclosed immunogenic polypeptide complex in combination with one or more other therapeutics. Likewise, also disclosed is the co-administration of one or more disclosed immunogenic polypeptide complex with one or more other therapeutics. Thus, the one or more disclosed immunogenic polypeptide complex can be administered simultaneously with one or more other therapeutics. Alternatively, a subject who is undergoing treatment with one or more other therapeutics can be administered the one or more disclosed immunogenic polypeptide complex as a co-therapy.

For example, researchers in Alzheimer's disease have identified several strategies as possible interventions against amyloid. β-Secretase inhibitors work to block the first cleavage of APP outside of the cell. γ-Secretase inhibitors (e.g., semagacestat) work to block the second cleavage of APP in the cell membrane and would then stop the subsequent formation of Aβ and its toxic fragments. Thus, the disclosed composition can include one or more of a β-Secretase inhibitor, a γ-secretase inhibitor (e.g., semagacestat), or a combination thereof.

Selective Aβ₄₂ lowering agents (e.g. tarenflurbil) modulate γ-secretase to reduce Aβ₄₂ production in favor of other (shorter) Aβ versions. Thus, the disclosed composition can include tarenflurbil.

Anti-aggregation agents such as apomorphine prevent Aβ fragments from aggregating or clear aggregates once they are formed. Thus, the disclosed composition can include apomorphine.

There is some indication that supplementation of the hormone melatonin may be effective against amyloid. This connection with melatonin, which regulates sleep, is strengthened by the recent research showing that the wakefulness inducing hormone orexin influences amyloid beta. Thus, the disclosed composition can include melatonin.

HU-210 is a synthetic cannabinoid that was first synthesized in 1988 by the group led by Professor Raphael Mechoulam at the Hebrew University. HU-210 is 1 to 80000 times more potent than natural THC from cannabis and has an extended duration of action. HU-210 is the (−)-1,1-dimethylheptyl analog of 11-hydroxy-Δ8-tetrahydrocannabinol, in some references it is called 1,1-dimethylheptyl-11-hydroxytetrahydrocannabinol. Per a 2005 article in the Journal of Clinical Investigation, HU-210 promotes proliferation, but not differentiation, of cultured embryonic hippocampal NS/PCs likely via a sequential activation of CB1 receptors, G(i/o) proteins, and ERK signaling. It was also indicated by this increased neural growth to entail antianxiety and antidepressant effects. Thus, the disclosed composition can include HU-210.

D. Source of Peptides

In some embodiments, the disclosed polypeptides are purified from human plasma using conventional techniques, such as, for example, antibodies that specifically bind the polypeptides, chromatography, gel electrophoresis, and the like.

In some embodiments, the disclosed immunogenic polypeptides are synthetic. In these embodiments, one or more of the amino acids of the polypeptide are linked together using conventional protein chemistry techniques.

In some embodiments, the disclosed immunogenic polypeptides are recombinant. In these embodiments, the polypeptides are produced by culturing a cell that expresses a nucleic acid encoding the polypeptide. The nucleic acid can be operably linked to an expression control sequence under conditions suitable for the transcription and translation of the nucleic acid.

E. Variants

Also disclosed are immunogenic variants of the disclosed polypeptides. Substitutions, deletions, insertions or any combination thereof may be combined to arrive at a final construct.

Insertions include amino and/or carboxyl terminal fusions as well as intra-sequence insertions of single or multiple amino acid residues. Insertions ordinarily will be smaller insertions than those of amino or carboxyl terminal fusions, for example, on the order of one to four residues.

Deletions are characterized by the removal of one or more amino acid residues from the protein sequence. Thus, the polypeptide can have 1, 2, 3, or 4 deletions from SEQ ID NO:1 or 5. These variants ordinarily are prepared by site specific mutagenesis of nucleotides in the DNA encoding the protein, thereby producing DNA encoding the variant, and thereafter expressing the DNA in recombinant cell culture.

Substitutional variants are those in which at least one residue has been removed and a different residue inserted in its place. Thus, the polypeptide can also have 1, 2, 3, or 4 substitutions within SEQ ID NO:1 or 5. Techniques for making substitution mutations at predetermined sites in DNA having a known sequence are well known, for example M13 primer mutagenesis and PCR mutagenesis.

1. Conservative Substitutions

In certain embodiments, the protein variant has a conservative amino acid substitution in SEQ ID NO:1 or 5. The replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr.

In contrast, the substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

2. Percent Identity

It is understood that one way to define the variants and derivatives of the disclosed polypeptides disclosed herein is through defining the variants and derivatives in terms of homology/identity to specific known sequences. Thus, disclosed are variants of these and other disclosed proteins. For example, disclosed are polypeptides having at least 20, 25, 30, 35, 40 amino acids in SEQ ID NO:1. For example, disclosed are polypeptides having at least 15, 20, 25, 30 amino acids in SEQ ID NO:5. Thus, disclosed are polypeptides having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:1 or 5.

Those of skill in the art readily understand how to determine the sequence identity of two proteins. For example, the sequence identity can be calculated after aligning the two sequences so that the sequence identity is at its highest level.

Another way of calculating sequence identity can be performed by published algorithms. Optimal alignment of sequences for comparison may be conducted by the local sequence identity algorithm of Smith and Waterman Adv. Appl. Math. 2: 482 (1981), by the sequence identity alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

It is understood that the description of conservative mutations and sequence identity can be combined together in any combination, such as embodiments that have at least 75% sequence identity to a particular sequence wherein the variants are conservative mutations.

3. Analogs and Mimetics

It is understood that there are numerous amino acid and peptide analogs which can be incorporated into the disclosed compositions. Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

Thus, also disclosed is a peptidomimetic of the disclosed immunogenic polypeptides. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment can be a peptidomimetic wherein the compound has a bond, a peptide backbone or an amino acid component replaced with a suitable mimic.

### a. Non-Natural Amino Acids

Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

There are also numerous D amino acids or amino acids which have a different functional substituent than natural amino acids. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Biotechnology, 12:678-682 (1994) all of which are herein incorporated by reference at least for material related to amino acid analogs). D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides.

### b. Modified Amino Acid Linkages

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, CH(OH)CH₂—, and —CHH₂SO— (these and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications; Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as β-alanine, γ-aminobutyric acid, and the like.

Cysteine residues can also be used to cyclize or attach two or more peptides together. This can be beneficial to constrain peptides into particular conformations. (Rizo and Gierasch Ann. Rev. Biochem. 61:387 (1992), incorporated herein by reference).

F. Multivalent Peptides

The disclosed polypeptides can be linked together to form divalent or multivalent peptides. In some embodiments, the polypeptides are directly linked together to form a polymer. Thus, disclosed is a polypeptide having two or more immunogenic polypeptide sequences. Thus, disclosed is a polypeptide having two or more amino acid sequences set forth in SEQ ID NO:1. Thus, disclosed is a polypeptide having two or more amino acid sequences set forth in SEQ ID NO:5. Also disclosed is a complex of divalent or multivalent polypeptides.

Two or more of the disclosed polypeptides can be linked together to form a conjugate. For example, disclosed is a composition including a first polypeptide having the amino acid sequence SEQ ID NO:1 or 5, or a conservative substitution or deletion thereof, and a second polypeptide having the amino acid sequence SEQ ID NO:1 or 5, or a conservative substitution or deletion thereof, wherein the first and second polypeptides are conjugated together with a linker. The linker can be any molecule, compound, or composition capable of joining two or more polypeptides together. For example, the linker can be one or more amino acids. The linker can be a polymer, such as polyethylene glycol (PEG).

Thus, in some embodiments, the polypeptides are linked to form a dendrimer. Peptide dendrimers are branched, often highly branched, artificial proteins in which several peptide chains branch out from a dendritic core matrix that is built up through the propagation of, for example, a trifunctional amino acid, such as Lys. Originally conceived as Multiple Antigen Presentation System (MAPS) for vaccine development, these molecules are also useful for protein design.

G. Pharmaceutical Compositions

Pharmaceutical compositions including a disclosed immunogenic polypeptide complex are provided.

The pharmaceutical composition can include an effective amount of an immunogenic polypeptide complex in a pharmaceutically acceptable excipient, wherein the immunogenic polypeptide complex includes an Aβ₄₂ polypeptide and an immunogenic fragment of RAGE, wherein the immunogenic fragment of RAGE can bind Aβ₄₂. Preferably, the Aβ₄₂ polypeptide and the immunogenic fragment of RAGE are in equimolar amounts. The term “equimolar amounts” includes up to 10% variation. Thus, equimolar amounts of the peptides includes a 45:55 ratio of the polypeptides. However, other suitable ratios of polypeptides can be used. For example, the Aβ₄₂ polypeptide and the immunogenic fragment of RAGE can be present in the immunogenic polypeptide complex in a 20:80, 25:75, 30:70, 35:65, 40:60, 50:50, 55:45, 60:40, 65:35, 70:30, 75:25, or 80:20 ratio, or any ratio in between.

Pharmaceutical compositions containing peptides or polypeptides may be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration. The compositions may also be administered using bioerodible inserts and may be delivered directly to an appropriate lymphoid tissue (e.g., spleen, lymph node, or mucosal-associated lymphoid tissue) or directly to an organ or tumor. The compositions can be formulated in dosage forms appropriate for each route of administration.

The precise dosage will vary according to a variety of factors such as subject-dependent variables (e.g., age, immune system health, etc.), the disease, and the treatment being effected. Therapeutically effective amounts of a immunogenic polypeptide complex provoke an immune response, which can result in the activation of lymphocytes, the secretion of antibodies, or a combination thereof.

In a preferred embodiment, the immunogenic polypeptide complex is administered in a range of 0.1-20 mg/kg based on extrapolation from tumor modeling and bioavailability. A most preferred range is 5-20 mg of immunogenic polypeptide complex/kg. Generally, for intravenous injection or infusion, dosage may be lower than when administered by an alternative route.

1. Formulations for Enteral Administration

In a preferred embodiment, the disclosed compositions, including those containing peptides and polypeptides, are formulated for oral (Enteral) delivery. Oral solid dosage forms are known to those skilled in the art. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 21st Ed. (2005, Lippincott, Williams & Wilins, Baltimore, Md. 21201) pages 889-964. The compositions may be prepared in liquid form, or may be in dried powder (e.g., lyophilized) form. Liposomal or polymeric encapsulation may be used to formulate the compositions. See also Marshall, K. In: Modern Pharmaceutics Edited by G. S. Banker and C. T. Rhodes Chapter 10, 1979. In general, the formulation will include the active agent and inert ingredients which protect the immunogenic polypeptide complex in the stomach environment, and release of the biologically active material in the intestine.

Liquid dosage forms for oral administration, including pharmaceutically acceptable emulsions, solutions, suspensions, and syrups, may contain other components including inert diluents; adjuvants such as wetting agents, emulsifying and suspending agents; and sweetening, flavoring, and perfuming agents.

2. Formulations for Parenteral Administration

The disclosed compositions, including those containing peptides and polypeptides, can also be administered in an aqueous solution for parental administration. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions include sterile water, buffered saline (e.g., Tris-HCl, acetate, phosphate), pH and ionic strength; and optionally, additives such as detergents and solubilizing agents (e.g., TWEEN® 20, TWEEN 80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

3. Controlled Delivery Polymeric Matrices

Compositions containing one or more immunogenic polypeptide complex or nucleic acids encoding the immunogenic polypeptide complex can be administered in controlled release formulations. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel. The matrix can also be incorporated into or onto a medical device to modulate an immune response, to prevent infection in an immunocompromised patient (such as an elderly person in which a catheter has been inserted or a premature child) or to aid in healing, as in the case of a matrix used to facilitate healing of pressure sores, decubitis ulcers, etc.

Either non-biodegradable or biodegradable matrices can be used for delivery of immunogenic polypeptide complex or nucleic acids encoding them, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

Controlled release oral formulations may be desirable. Immunogenic polypeptide complex can be incorporated into an inert matrix which permits release by either diffusion or leaching mechanisms, e.g., films or gums. Slowly disintegrating matrices may also be incorporated into the formulation. Another form of a controlled release is one in which the drug is enclosed in a semipermeable membrane which allows water to enter and push drug out through a single small opening due to osmotic effects. For oral formulations, the location of release may be the stomach, the small intestine (the duodenum, the jejunem, or the ileum), or the large intestine. Preferably, the release will avoid the deleterious effects of the stomach environment, either by protection of the active agent (or derivative) or by release of the active agent beyond the stomach environment, such as in the intestine. To ensure full gastric resistance an enteric coating (i.e, impermeable to at least pH 5.0) is essential. These coatings may be used as mixed films or as capsules such as those available from Banner Pharmacaps.

4. Liposomes

Also disclosed is a pharmaceutical composition having an effective amount of one or more disclosed immunogenic polypeptide complex in a liposome. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. These MLVs were first described by Bangham, et al., J Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other fowls of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamnellar vesicles, which are prepared by more extensive sonication of MLVs, and are made of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 μm, are described in Callo, et al., Cryobiology 22(3):251-267 (1985).

Mezei, et al., U.S. Pat. Nos. 4,485,054 and 4,761,288 also describe methods of preparing lipid vesicles. More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use in the invention are incorporated herein by reference.

Fatty acids (i.e., lipids) that can be conjugated to the provided compositions include those that allow the efficient incorporation of the disclosed compositions into liposomes. Generally, the fatty acid is a polar lipid. Thus, the fatty acid can be a phospholipid. The provided compositions can include either natural or synthetic phospholipid. The phospholipids can be selected from phospholipids containing saturated or unsaturated mono or disubstituted fatty acids and combinations thereof. These phospholipids can be dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidie acid, palmitelaidoyloleoylphosphatidylcholine, palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinoleoylphosphatidic acid. These phospholipids may also be the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in these lysophosphatidyl derivatives may be palimtoyl, oleoyl, palmitoleoyl, linoleoyl myristoyl or myristoleoyl. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can include palmitoyl 16:0.

5. Excipents

The compositions disclosed can be used therapeutically in combination with a pharmaceutically acceptable excipient/carrier. Thus, also disclosed is a pharmaceutical composition having an effective amount of one or more disclosed immunogenic polypeptide complex and a pharmaceutically acceptable excipient.

Pharmaceutical excipients are known to those skilled in the art. These most typically would be standard carriers for administration of drugs to humans, including solutions such as sterile water, saline, and buffered solutions at physiological pH. Pharmaceutical compositions may include carriers, thickeners, diluents, buffers, preservatives, surface active agents and the like in addition to the active agent. Pharmaceutical compositions may also include one or more active ingredients such as antimicrobial agents, anti-inflammatory agents, anesthetics, and the like.

Suitable pharmaceutical preparations include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Suitable formulations include sprays, liquids and powders. Conventional pharmaceutical carriers, aqueous, powder or oily bases, thickeners and the like may be necessary or desirable. Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

H. Vaccines Including Immunogenic Polypeptide Complex

The disclosed immunogenic polypeptide complex can be administered as a component of a vaccine to induce an immune response. Vaccines disclosed herein include immunogenic polypeptide complex and optionally adjuvants and targeting molecules. Sources of immunogenic polypeptide include any disclosed polypeptides, fusion proteins thereof, variants thereof, nucleic acids encoding these polypeptides and fusion proteins, or variants thereof or host cells containing vectors that express the immunogenic polypeptides.

Optionally, the vaccines described herein may include adjuvants. The adjuvant can be, but is not limited to, one or more of the following: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor. Such proteinaceous adjuvants may be provided as the full-length polypeptide or an active fragment thereof, or in the form of DNA, such as plasmid DNA.

I. Nucleic Acids

1. Nucleic Acids Encoding the Peptides

Also disclosed are nucleic acids encoding the disclosed polypeptides. Thus, disclosed are all nucleic acids, including degenerate nucleic acids, encoding the disclosed variants and derivatives of the protein sequences. While each particular nucleic acid sequence may not be written out, it is understood that each and every sequence is in fact disclosed and described through the disclosed protein sequence.

2. Expression Control Sequences

The nucleic acids that are delivered to cells typically contain expression control systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements. Thus, also disclosed are nucleic acids encoding the disclosed polypeptides operably linked to an expression control sequence.

Promoters, enhancers, transcriptional and translational stop sites, and other signal sequences are examples of nucleic acid sequences operatively linked to other sequences. For example, operative or operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P .J. et al., Gene 18: 355-360 (1982)). Of course, promoters from the host cell or related species can also be used.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 by in length. Enhancers function to increase transcription from nearby promoters. Enhancers also often contain response elements that mediate the regulation of transcription. Promoters can also contain response elements that mediate the regulation of transcription.

Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and contains of about 400 bases. It is also preferred that the transcribed units contain other standard sequences alone or in combination with the above sequences improve expression from, or stability of, the construct.

3. Vectors Containing the Nucleic Acids

Also disclosed is a vector containing a nucleic acid encoding the disclosed polypeptides. In some embodiments the vector is derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. One embodiment is a viral vector which has been engineered so as to suppress the immune response of the host organism, elicited by the viral antigens.

4. Cells Containing Vectors

Also disclosed are cells containing one or more of the disclosed nucleic acids or vectors. A cell culture can be a population of cells grown on a medium such as agar. A primary cell culture is a culture from a cell or taken directly from a living organism, which is not immortalized.

### III. Methods of Manufacture

A. Methods for Producing Immunogenic Polypeptides

Isolated immunogenic polypeptides, variants thereof, and fusion proteins thereof can be obtained by, for example, chemical synthesis or by recombinant production in a host cell. To recombinantly produce an immunogenic polypeptide, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding an immunogenic polypeptide. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well know in the art include, for example, Escherichia coli strains such as BL-21, and cultured mammalian cells such as CHO cells.

In eukaryotic host cells, a number of viral-based expression systems can be utilized to express immunogenic polypeptides. Viral based expression systems are well known in the art and include, but are not limited to, baculoviral, SV40, retroviral, or vaccinia based viral vectors.

Mammalian cell lines that stably express immunogenic polypeptides can be produced using expression vectors with appropriate control elements and a selectable marker. For example, the eukaryotic expression vectors pCR3.1 (Invitrogen Life Technologies) and p91023(B) (Wong et al. (1985) Science 228:810-815) are suitable for expression of immunogenic polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, the immunogenic polypeptide can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Immunogenic polypeptides can be isolated using, for example, chromatographic methods such as DEAE ion exchange, gel filtration, and hydroxylapatite chromatography. For example, a costimulatory polypeptide in a cell culture supernatant or a cytoplasmic extract can be isolated using a protein G column. In some embodiments, variant costimulatory polypeptides can be “engineered” to contain an amino acid sequence that allows the polypeptides to be captured onto an affinity matrix. For example, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify immunogenic polypeptides.

Methods for introducing random mutations to produce variant polypeptides are known in the art. Random peptide display libraries can be used to screen for immunogenic polypeptides. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially.

B. Methods for Producing Isolated Nucleic Acid Molecules Encoding Immunogenic Polypeptides

Isolated nucleic acid molecules encoding immunogenic polypeptides can be produced by standard techniques, including, without limitation, common molecular cloning and chemical nucleic acid synthesis techniques. For example, polymerase chain reaction (PCR) techniques can be used to obtain an isolated nucleic acid encoding an immunogenic polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. Immunogenic polypeptide-encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992. Examples of amino acid positions that can be modified include those described herein.

C. Complex Formation

The disclosed immunogenic polypeptide complex can be formed using routine methods. For example, equimolar amounts of the first and second immunogenic polypeptides can be incubated in solution under physiological conditions to promote complex formation. As an example, Aβ₄₂-RAGE complex was formed by incubating equimolar amounts of Aβ₄₂ and an immunogenic fragment of RAGE (RAGE₂₃₋₅₄) for one month in sterile water at 37° C.

In some embodiments, the immunogenic polypeptide complex is formed using a protein crosslinker. Protein crosslinkers that can be used to crosslink the immunogenic polypeptides are known in the art and are defined based on utility and structure and include DSS (Disuccinimidylsuberate), DSP (Dithiobis(succinimidylpropionate)), DTSSP (3,3′-Dithiobis (sulfosuccinimidylpropionate)), SULFO BSOCOES (Bis[2-(sulfosuccinimdooxycarbonyloxy)ethyl]sulfone), BSOCOES (Bis[2-(succinimdooxycarbonyloxy)ethyl]sulfone), SULFO DST (Disulfosuccinimdyltartrate), DST (Disuccinimdyltartrate), SULFO EGS (Ethylene glycolbis(succinimidylsuccinate)), EGS (Ethylene glycolbis(sulfosuccinimidylsuccinate)), DPDPB (1,2-Di[3′-(2′-pyridyldithio)propionamido]butane), BSSS (Bis(sulfosuccinimdyl) suberate), SMPB (Succinimdyl-4-(p-maleimidophenyl) butyrate), SULFO SMPB (Sulfosuccinimdyl-4-(p-maleimidophenyl) butyrate), MBS (3-Maleimidobenzoyl-N-hydroxysuccinimide ester), SULFO MBS (3-Maleimidobenzoyl-N-hydroxysulfosuccinimide ester), STAB (N-Succinimidyl(4-iodoacetyl) aminobenzoate), SULFO STAB (N-Sulfosuccinimidyl(4-iodoacetyl)aminobenzoate), SMCC (Succinimidyl-4-(N-maleimidomethyl)cyclohexane-1-carboxylate), SULFO SMCC (Sulfosuccinimidyl-4-(N-maleimidomethyl)cyclohexane-1-carboxylate), NHS LC SPDP (Succinimidyl-6-[3-(2-pyridyldithio)propionamido)hexanoate), SULFO NHS LC SPDP (Sulfosuccinimidyl-6-[3-(2-pyridyldithio)propionamido)hexanoate), SPDP (N-Succininadyl-3-(2-pyridyldithio)propionate), NHS BROMOACETATE (N-Hydroxysuccinimidylbromoacetate), NHS IODOACETATE (N-Hydroxysuccinimidyliodoacetate), MPBH (4-(N-Maleimidophenyl)butyric acid hydrazide hydrochloride), MCCH (4-(N-Maleimidomethyl)cyclohexane-1 -carboxylic acid hydrazide hydrochloride), MBH (m-Maleimidobenzoic acid hydrazidehydrochloride), SULFO EMCS (N-(epsilon-Maleimidocaproyloxy)sulfosuccinimide), EMCS (N-(epsilon-Maleimidocaproyloxy)succinimide), PMPI (N-(p-Maleimidophenyl)isocyanate), KMUH (N-(kappa-Maleimidoundecanoic acid) hydrazide), LC SMCC (Succinimidyl-4-(N-maleimidomethyl)-cyclohexane-1-carboxy(6-amidocaproate)), SULFO GMBS (N-(gamma-Maleimidobutryloxy) sulfosuccinimide ester), SMPH (Succinimidyl-6-(beta-maleimidopropionamidohexanoate)), SULFO KMUS (N-(kappa-Maleimidoundecanoyloxy)sulfosuccinimide ester), GMBS (N-(gamma-Maleimidobutyrloxy)succinimide), DMP (Dimethylpimelimidate hydrochloride), DMS (Dimethylsuberimidate hydrochloride), MHBH(Wood's Reagent) (Methyl-p-hydroxybenzimidate hydrochloride, 98%), DMA (Dimethyladipimidate hydrochloride).

### IV. Methods of Use

A. Activation of Lymphocytes

The disclosed immunogenic polypeptide complex can be used to activate lymphocytes. For example, the disclosed immunogenic polypeptide complex of Aβ and RAGE can be used to activate B lymphocytes (B cells) that secrete antibodies that specifically bind Aβ₄₂, RAGE, or the [Aβ₄₂-RAGE] complex.

In some embodiments, the disclosed immunogenic polypeptide complex can be used to activate T cells (i.e., increase antigen-specific proliferation of T cells, enhance cytokine production by T cells, stimulate differentiation and effector functions of T cells and/or promote T cell survival).

Methods for using immunogenic polypeptide complex include contacting a lymphocyte with an immunogenic polypeptide complex in an amount effective to invoke an immune response. The contacting can be in vitro, ex vivo, or in vivo (e.g., in a mammal such as a mouse, rat, rabbit, dog, cow, pig, non-human primate, or a human).

Thus, provided is a method of provoking an immune response in a lymphocyte, involving contacting the lymphocyte with an immunogenic polypeptide complex of Aβ₄₂ polypeptide and an immunogenic fragment of RAGE, wherein the immunogenic fragment of RAGE can bind Aβ₄₂. In some embodiments, the immunogenic fragment of RAGE has an amino acid sequence of at least 20 amino acids having at least 95% sequence identity to SEQ ID NO:2. In some embodiments, the immunogenic fragment of RAGE comprises an amino acid sequence having at least 95% sequence identity to SEQ ID NO:5. Thus, in some embodiments, the immunogenic fragment of RAGE comprises the amino acid sequence SEQ ID NO:5, or a conservative variant thereof.

In some embodiments, the lymphocyte is in a human subject. In other embodiments, the lymphocyte is in a non-human subject. In still other embodiments, the lymphocyte is ex vivo.

In some embodiments, the immunogenic polypeptide complex can be administered directly to a lymphocyte. Alternatively, an APC such as a macrophage, monocyte, interdigitating dendritic cell (referred to herein as a dendritic cell), or B cell can be transformed, transduced, or transfected with a nucleic acid containing a nucleotide sequence that encodes an immunogenic polypeptide complex, and the lymphocyte can be contacted by the transformed, transduced, or transfected APC. The transformed, transduced, or transfected cell can be a cell, or a progeny of a cell that, prior to being transformed, transduced, or transfected, can be obtained from the subject to which it is administered, or from another subject (e.g., another subject of the same species).

The immunogenic polypeptide complex can be any immunogenic polypeptide complex described herein. The immunogenic polypeptide complex of these methods can be in a pharmaceutically acceptable carrier.

If the activation is in vitro, the immunogenic polypeptide complex can be bound to the floor of a relevant culture vessel, or bead or other solid support, e.g. a well of a plastic microtiter plate.

In vitro application of the immunogenic polypeptide complex can be useful, for example, in basic scientific studies of immune mechanisms or for production of activated lymphocytes for use in studies of lymphocytes function or, for example, passive immunotherapy. Furthermore, immunogenic polypeptide complex can be added to in vitro assays (e.g., lymphocyte proliferation assays) designed to test for immunity to an antigen of interest in a subject from which the lymphocytes were obtained. Addition of immunogenic polypeptide complex to such assays would be expected to result in a more potent, and therefore more readily detectable, in vitro response.

B. Methods of Stimulating an Immune Response

The disclosed immunogenic polypeptide complex is generally useful in vivo and ex vivo as a therapeutic for stimulating an immune response. In general, the disclosed immunogenic polypeptide complex is useful for treating a subject having or being predisposed to any disease or disorder to which the subject's immune system mounts an immune response. The disclosed compositions are useful to stimulate or enhance humoral and/or cell-mediated immune responses in a subject.

The disclosed immunogenic polypeptide complex is useful for stimulating or enhancing an immune response in a host by administering to subject an amount of the immunogenic polypeptide complex effective to stimulate an immune response in the subject.

The disclosed immunogenic polypeptide complex or nucleic acids encoding the same may be administered alone or in combination with any other suitable treatment. In one embodiment the immunogenic polypeptide complex can be administered in conjunction with, or as a component of, a vaccine composition. Suitable components of vaccine compositions are described above. The disclosed immunogenic polypeptide complex can be administered prior to, concurrently with, or after the administration of another vaccine, such as an Alzheimer's vaccine.

The desired outcome of the immune response may vary according to the disease, according to principles well known in the art. Immune responses may completely treat a disease, may alleviate symptoms, or may be one facet in an overall therapeutic intervention against a disease.

C. Methods of Stabilizing Antigens for Oral Vaccine Delivery

1. Amyloid Beta

The disclosed examples demonstrate the use of complex of Aβ₄₂ as a method of orally delivering antigens to the immune system. These data support that this method produces antibodies in the plasma and brain of animals within 16 weeks. In addition, the disclosed protein complex is very stable. This indicates that these may be stable for quite some time without refrigeration. A major hurdle in vaccination in the non-industrialized nations of the world is that vaccines are required to be refrigerated throughout the entire distribution process. This stability problem has hampered the use of some very effective vaccines from effectively reaching target populations.

It has been shown that RAGE plays an important role in fueling neurotoxic Aβ₄₂ plaques in AD brain. One important consequence of the RAGE-Aβ interaction is the RAGE-dependent transport of Aβ from blood to brain. Disclosed is a second consequence of the RAGE-Aβ interaction—the formation of a soluble high molecular weight complex of the two proteins that is both neurotoxic and immunogenic. AGEs in presence of Aβ enhance APP expression thereby enhancing Aβ release, and Aβ doubles its aggregation by two dominant receptors (RAGE and α7nACRs). It has been shown that Aβ counter-influences its binding receptor (RAGE or α7nACRs) to support its aggregation, unlike other ligands binding to RAGE, such as AGEs, which are eliminated by RAGE. The unforeseen autoimmune-like response observed in mice immunized with human neurofilament AGEs, which produced higher concentrations of RAGE and Aβ₄₂, led to the discovery of a RAGE-Aβ complex immunogen circulating in blood plasma.

Thus, disclosed are methods of enhancing the immunogenic potential, stability, and immunogenicity of Aβ₄₂ and RAGE, involving aggregating immunogenic fragments of RAGE with Aβ₄₂ polypeptide. The Aβ₄₂-RAGE complex formed from this method can be used to produce an oral Alzheimer's vaccine.

It is understood that Aβ₄₂ stabilizes RAGE and RAGE supports Aβ₄₂ aggregation. Activated RAGE enhances inflammation, oxidation and neurodegeneration. It is shown that RAGE activation does not allow wound healing to occur and therefore the disclosed vaccine is ideal in suppressing RAGE expression levels on one hand and eliminating Aβ₄₂ on the other hand.

Thus, provided is a method of stabilizing a polypeptide antigen for production of an oral vaccine, the method involving complexing the polypeptide antigen with Aβ. As shown, the polypeptide antigen can be an immunogenic fragment of RAGE that can bind Aβ₄₂.

In some embodiments, the immunogenic fragment of RAGE comprises an amino acid sequence of at least 20 amino acids having at least 95% sequence identity to SEQ ID NO:2. In some embodiments, immunogenic fragment of RAGE has an amino acid sequence having at least 95% sequence identity to SEQ ID NO:5. In preferred embodiments, the immunogenic fragment of RAGE has the amino acid sequence SEQ ID NO:5, or a conservative variant thereof.

In preferred embodiments, the Aβ is Aβ₄₂ and comprises the amino acid sequence SEQ ID NO:1, or a conservative variant thereof. Thus, in a preferred embodiment, the Aβ consists of the amino acid sequence SEQ ID NO:1 and the polypeptide antigen consist of the amino acid sequence SEQ ID NO:5.

Based on the disclosed advantages of the Aβ₄₂-RAGE complex, it is also disclosed that Aβ₄₂ can be used as a delivery system for other antigens. For example, an Aβ polypeptide, such as Aβ₄₂, can be used as a delivery system for antigens relating to other diseases such as Hepatitis. These Aβ-antigen complexes can be stable at ambient temperatures.

It is understood that Aβ-RAGE complex creates an amyloid plaque that entrains the RAGE fragment, protecting the peptides on the interior from the harsh environment of the stomach. This in turn allows these two peptides to reach the antigen presenting cells in the intestines (Peyer's Patches). This approach can be a universal approach to vaccine delivery.

For example, Aβ polypeptide, such as Aβ₄₂, can be used in a Hepatitis vaccine to deliver amino acids 12-32 of the preS1 gene for the envelope protein of Hepatitis B. This amino acid sequence contains the recognition sites for both T-helper and B-cells. In addition, adding this 21 amino acid peptide to a 9 amino acid sequence from cytokine interleukin-1 (IL-1) via a diglycine linker dramatically improves antibody production in mice (Rao et al. 1990, PNAS 87: 5519-5522). This proposed 32 amino acid is of similar size to the RAGE antigen that was successfully complexed with Aβ₄₂. Thus, successful aggregation of the enhanced Hepatitis antigen with Aβ₄₂ can result in a viable oral vaccine candidate for Hepatitis B. This vaccine can have similar ambient temperature stability observed for Aβ-RAGE complex, and therefore represents an ability to dramatically change vaccination strategies in underdeveloped nations where Hepatitis is a significant health concern.

Sequence of enhanced Hepatitis B Antigen [SI (12-32)—diglycine linker—IL-1 (163-171)] with diglycine linker underlined is shown below:

MGTNLSVPNP LGFLPDHQLD PGGVQGEESN DK (SEQ ID NO:3).

2. Alternatives to Amyloid Beta for Stabilizing Antigens

One concern for the approach described above is that it would create Aβ antibodies in addition to potentially creating antibodies for HSB. One suitable candidate to replace Aβ₄₂ is amino acids 124-147 of the major Hepatitis 13 surface antigen (HBsAg) Protein. A sequence for cycteine-rich HBsAg amino acids 124-147 is set forth in SEQ ID NO:4, shown below:

CTTPAQGNSM FPSCCCTKPT DGNC (SEQ ID NO:4).

This cysteine-rich sequence spontaneously forms a disulfide linked polypeptide mass of 8-35 kDa in size (Manivel et al., 1992, J Immunol 148(12): 4006-4011). This aggregate illicits antibody production against Hepatitis B. The aggregation is enhanced, without the loss of immunostilulatory properties, by N-terminal myristylation (Manivel et al., 1993, Vaccine 11(3): 366-371). Therefore, this aggregate can be used as an oral vaccine alone, or in complex a enhanced Hepatitis antigen, such as the one shown in SEQ ID NO:1.

The present disclosure therefore provides a general delivery strategy for oral vaccines using polypeptide complex. This strategy can be applied using peptides such as Aβ₄₂ but can be expanded to other peptides that can form complex, induce antibody production, and possess appropriate stability in the gastrointestinal tract.

### Examples

### Example 1

The immune system was stimulated to produce antibodies against Aβ peptides, substances that are formed in the brain in individuals with AD, and which lead to the toxicity and degeneration of brain cells. The antibodies formed can lower the body's levels of free Aβ peptide, and those in complex with the RAGE. This approach can cause a gradual reduction in the toxic Aβ peptides and peptide complexes that form in the blood and brain of Alzheimer's patients; and as a result, stop or delay the progression of the disease.

Various means were studied to enhance the immune potential of amyloid peptides. One such approach was derived from experiments with samples of human plasma and brain tissues having a complex of peptides that expressed epitopes for both human Aβ peptide and human RAGE. This involved the incubation of equimolar amounts of Aβ and an immunogenic fragment of RAGE for one month in sterile water at 37° C.

After incubation, the solution was dialyzed to remove unused products. The dialyzed solution was then concentrated in a centrifugation filter with a 10,000 molecular weight cutoff. A sample of the concentrated material was subjected to 10% SDS gel electrophoresis where a single band at 120 kD was revealed. The concentrated complex was dissolved in water and administered orally to rats on three occasions with two weeks between administrations. Two weeks after the last administration plasma samples were obtained for the determination of Aβ or RAGE titers.

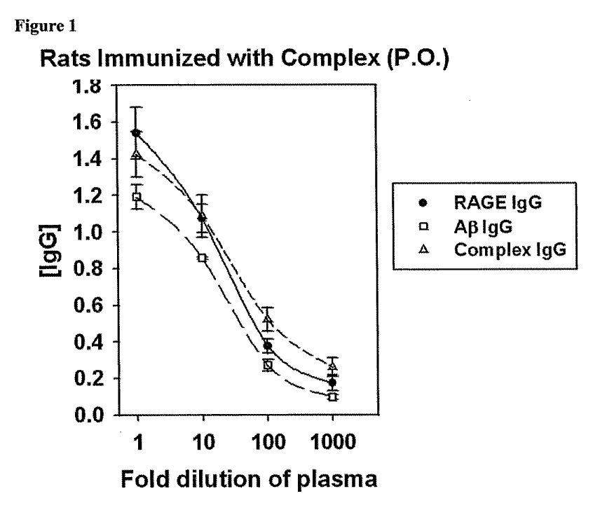

The data in FIG. 1 show the results of oral immunization with complex antigen in rats. A clear production of circulating anti-Aβ and anti-RAGE IgGs in rat plasma, as well as antibodies against the complex peptide. The titer for the latter was similar to that for RAGE and slightly greater than that for AS, indicating the existence of separate anti-complex antibodies. These studies provide indicate the potential for development of an orally-effective vaccine with for aiding the clearance of both free and bound or complexed amyloid peptides.

### Example 2

The B6C3-Tg (APPswe, PSEN1 dE9) 85Dbo/J mouse strain was used for the studies described below. This double transgenic mouse expresses mutant human presenilin 1 (DeltaE9) and a chimeric mouse/human APPswe mutations. The mouse prion promoter directs expression of both transgenes. The DeltaF9 mutation of the human presenilin 1 gene is a deletion of exon 9 and corresponds to a form of early-onset AD. The amyloid precursor protein is altered by “humanizing” the Aβ domain of the mouse coding sequence by replacing 3 amino acids that differ between the two species with the human residues. This allows mice to secrete a human Aβ peptide. Both the transgenic peptide, and holoprotein, can be detected by antibodies specific for human sequence for this region. The chimeric APP was then further modified to encode the Swedish mutations K595M/N596L in order to elevate the amount of Aβ produced by favoring processing through the y-secretase pathway. A high level of human presenilin protein, which displaces detectable endogenous mouse protein, is also immunodetected in whole brain protein homogenates. Transgenic mice develop cerebral Aβ deposits by 6 to 7 months of age. Heterozygous breeding pairs were originally purchased from Jackson Laboratories, Maine USA; further breeding and maintenance of the colony was carried out in the Laboratory Animal Services facility at the Medical College of Georgia. Mice were maintained in a humidity (50-55%) and temperature (21-23° C.)-controlled room on a 12-h light/dark cycle (lights on at 6:30 AM). Genotyping was performed in-house by standard procedures.

In a second series of experiments double transgenic AD mice (APPSWE-PS 1) at ages from 3-18 months old were administered the complex antigen by oral gavage periodically over a period of 16 weeks. Plasma samples were subjected to ammonium sulfate precipitation followed by dialysis and purification on a protein G column to isolate an IgG fraction. This purified IgG fraction was used in an ELISA procedure to determine the titers of anti-RAGE and anti-Aβ IgGs. In addition, for the rat plasma samples, ELISA plate wells were coated with the complex peptide, and the purified IgG fraction was probed for anti-complex antibodies.

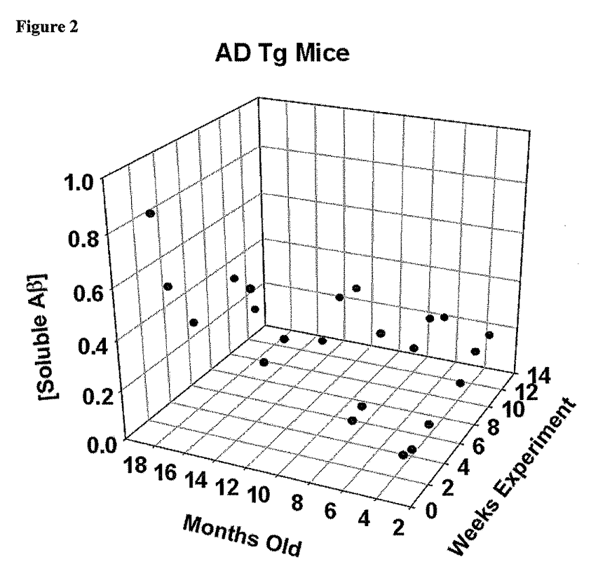

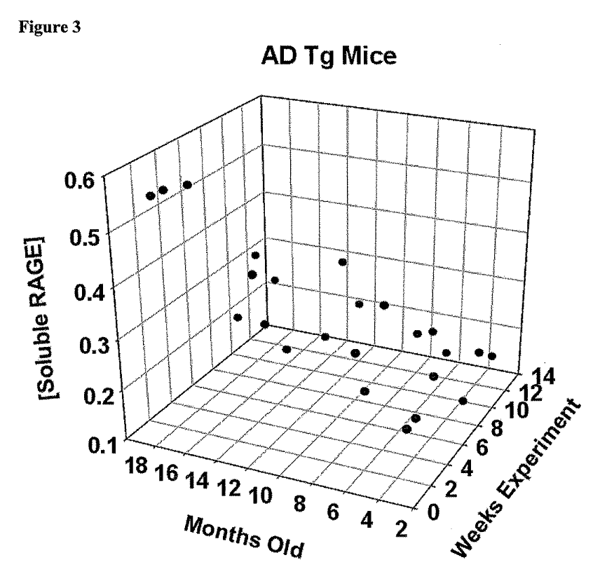

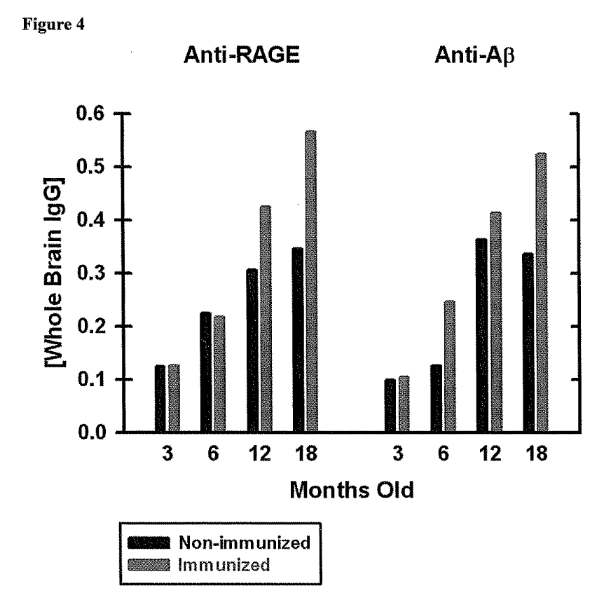

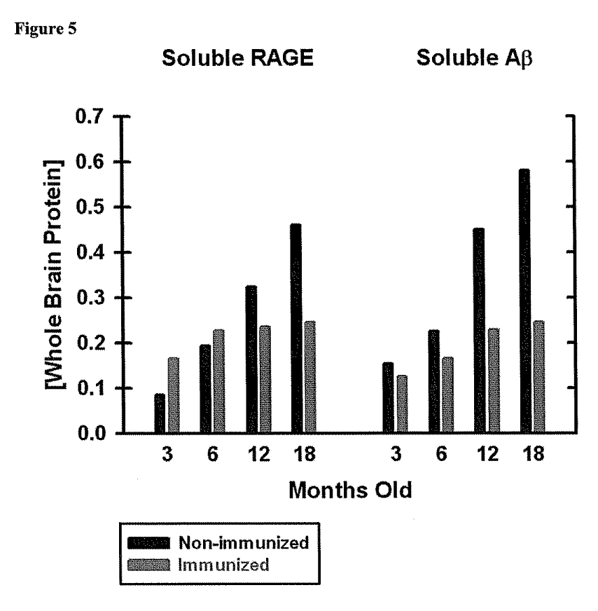

These experiments showed an age-dependent increase in Aβ and RAGE soluble peptides in the plasma of the AD Tg non-immunized mice (FIG. 5). The experiments also show the ability of immunization by oral gavage with the complex antigen to decrease plasma levels of both peptides, particularly for the oldest (18 months) group of animals (FIG. 5). Immunization with the complex antigen subsequently resulted in a time-dependent decrease in brain levels of anti-Aβ and anti-RAGE IgG (FIGS. 2, 3, and 4).

The data support the ability of these antibodies to enter the CNS, though the levels were not quite as high as in the plasma. However, active immunization has been suggested to act by at least two potential mechanisms. The sink hypothesis is based on the ability of anti-Aβ antibodies to increase the clearance of plasma Aβ. This is followed by dissolution of cerebral amyloid plaques which are in equilibrium with CSF amyloid, which in turn is in equilibrium with plasma amyloid. The other mechanism takes advantage of the reduction in the integrity of the blood-brain-barrier in AD with the passage of antibody molecules into the brain.

Though the relative contribution of each of these potential mechanisms to the present results is not clear, the data shown in FIG. 5 demonstrates that the orally administered complex antigen results in the decrease of brain Aβ and RAGE levels, particularly for the two oldest groups of animals measured at the completion of the immunization period (16 weeks). Immunization also prevented the accumulation of brain amyloid with age in younger animals. These results provide clear proof of principal in one of the most accepted models for AD.

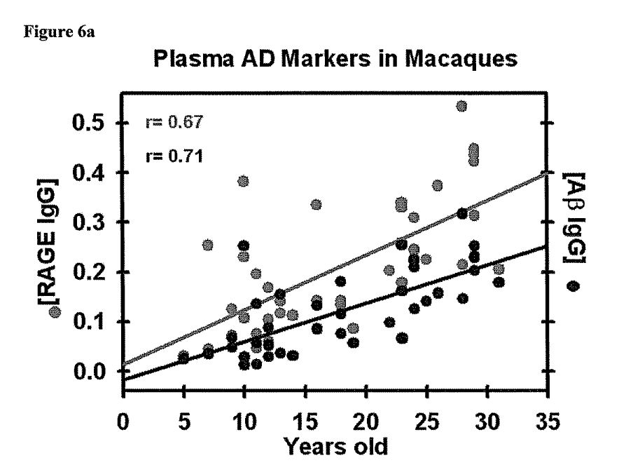

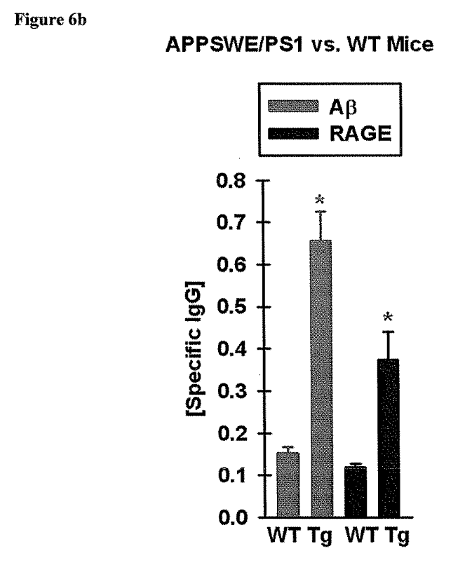

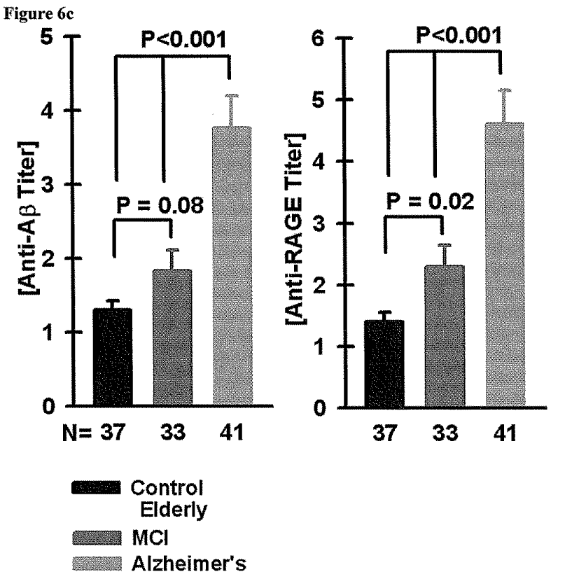

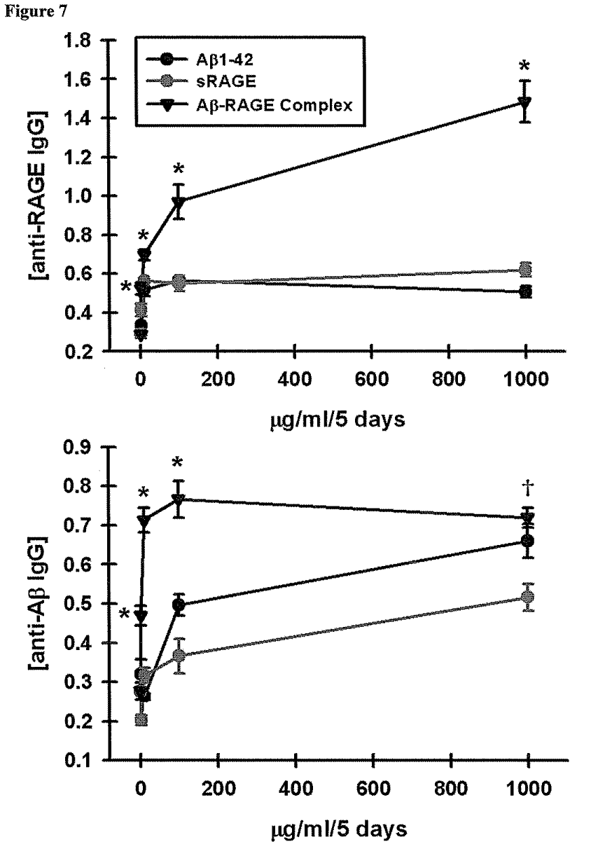

As shown in FIG. 6a, plasma anti-RAGE and anti-Aβ IgGs increase with age in macaque monkeys of varying ages. Likewise, plasma anti-RAGE and anti-Aβ IgGs are increased in AD transgenic (APPSWE/PS1) mice (FIG. 6b). Finally, plasma anti-RAGE and anti-Aβ IgGs are elevated in human subjects with mild-cognitive impairment and even more so in patients with Alzheimer's disease (FIG. 6c). Finally, the RAGE/Aβ complex antigen produced a more rapid and significantly greater quantity of plasma anti-RAGE and anti-Aβ IgGs as compared to Aβ₄₂ or sRAGE alone (FIG. 7).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A pharmaceutical composition comprising an effective amount of an immunogenic polypeptide complex in a pharmaceutically acceptable excipient, wherein the immunogenic polypeptide complex comprises an amyloid beta 42 (Aβ₄₂) polypeptide and an immunogenic fragment of receptor for advanced glycation endproducts (RAGE), wherein the immunogenic fragment of RAGE can bind Aβ₄₂.

2. The pharmaceutical composition of claim 1, wherein the Aβ₄₂ polypeptide and the immunogenic fragment of RAGE are in approximately equimolar amounts.

3. The pharmaceutical composition of claim 1, wherein the immunogenic fragment of RAGE comprises an amino acid sequence having at least 95% sequence identity to SEQ ID NO:5.

4. The pharmaceutical composition of claim 3, wherein the immunogenic fragment of RAGE comprises the amino acid sequence SEQ ID NO:5, or a conservative variant thereof.

5. A method of stabilizing a polypeptide antigen for production of an oral vaccine, the method comprising complexing the polypeptide antigen with an amyloid beta (Aβ) polyeptide.

6. The method of claim 5, wherein the polypeptide antigen is an immunogenic fragment of receptor for advanced glycation endproducts (RAGE) that can bind Aβ₄₂.

7. The method of claim 6, wherein the immunogenic fragment of RAGE comprises an amino acid sequence of at least 20 amino acids having at least 95% sequence identity to SEQ ID NO:2.

8. The method of claim 7, wherein the immunogenic fragment of RAGE comprises an amino acid sequence having at least 95% sequence identity to SEQ ID NO:5.

9. The method of claim 8, wherein the immunogenic fragment of RAGE comprises the amino acid sequence SEQ ID NO:5, or a conservative variant thereof.

10. The method of claim 5, wherein the Aβ polypeptide is Aβ₄₂ and comprises the amino acid sequence SEQ ID NO:1, or a conservative variant thereof.

11. The method of claim 5, wherein the Aβ polypeptide consists of the amino acid sequence SEQ ID NO:1 and the polypeptide antigen consist of the amino acid sequence SEQ ID NO:5.

12. A method of provoking an immune response in a lymphocyte, comprising contacting the lymphocyte with an immunogenic polypeptide complex of amyloid beta 42 (Aβ₄₂) polypeptide and an immunogenic fragment of RAGE, wherein the immunogenic fragment of RAGE can bind Aβ₄₂.

13. The method of claim 12, wherein the immunogenic fragment of RAGE comprises an amino acid sequence of at least 20 amino acids having at least 95% sequence identity to SEQ ID NO:2.

14. The method of claim 13, wherein the immunogenic fragment of RAGE comprises an amino acid sequence having at least 95% sequence identity to SEQ ID NO:5,

15. The method of claim 14, wherein the immunogenic fragment of RAGE comprises the amino acid sequence SEQ ID NO:5, or a conservative variant thereof.

16. The method of claim 12, wherein the lymphocyte is in a human subject.

17. The method of claim 12, wherein the lymphocyte is in a non-human subject.

18. The method of claim 12, wherein the lymphocyte is ex vivo.

19. The method of claim 12, wherein the immunogenic polypeptide complex is in a pharmaceutically acceptable carrier.

20. A method of treating Alzheimer's disease in a subject, comprising administering to the subject the pharmaceutical composition of claim 1.
